(19) United States
(12) Patent Application Publication
(10) Pub. No.: US 2014/0234234 A1
(43) Pub. Date: Aug. 21, 2014

(54) DEODORANT AND ANTIPERSPIRANT COMPOSITION, METHOD FOR PRODUCING A DEODORANT AND ANTIPERSPIRANT COMPOSITION, AND METHOD FOR PREVENTING AND/OR REDUCING PERSPIRATION ODOUR

(71) Applicant: Luiz Francisco Pianowski, Braganca Paulista (BR)

(72) Inventor: Luiz Francisco Pianowski, Braganca Paulista (BR)

(73) Assignee: Kyolab Laboratorio Ltda, Campinas (BR)

(21) Appl. No.: 14/009,096

(22) PCT Filed: Mar. 19, 2012
(86) PCT No.: PCT/BR2012/000177

§ 371 Date: Sep. 30, 2013

(30) Foreign Application Priority Data
Mar. 31, 2011 (BR) PI1101172-6

(51) Int. Cl. A61K 8/97; A61Q 15/00

(52) U.S. Cl. CPC A61K 8/97; A61Q 15/00
USPC 424/59; 424/65

(57) ABSTRACT

The present invention relates to deodorant and antiperspirant compositions containing a natural active principle, containing tannins in the formulation. These compositions are suitable for use in all cosmetic formulations, such as sticks, aerosols, sprays and creams. The invention further relates to processes for production of deodorant and antiperspirant compositions, and to a method for prevention and/or reduction of perspiration odors.

### FIELD OF THE INVENTION

The present invention relates to deodorant and antiperspirant compositions containing natural active principle in the formulation, which are tannins. These compositions are suitable for use in all cosmetic formulations, such as sticks, aerosols, sprays and creams.

The invention further relates to processes for production of deodorant and antiperspirant compositions, and to a method for prevention and/or reduction of perspiration odors.

### BACKGROUND OF THE INVENTION

The use of topical compositions in order to absorb, reduce or eliminate generally unpleasant axillary odors, usually from perspiration is a common practice in the cosmetic field.

It is known that sweat (ecrine and apocrine) has little odor when secreted and that the degradation of sweat by bacteria present in the skin through enzymatic reactions is responsible for the production of compounds with unpleasant odor.

Generally, there are three major groups of compounds used to address body stench: perfumes, antiperspirants and deodorants.

Perfumes typically only disguise the stench by masking it; the antiperspirants block the sweat glands, thus reducing the perspiration; and deodorants use many microbial agents to control the amount of microorganisms present on the skin and reduce their metabolic activities as well.

There is a variety of substances used to control bacterial flora resident on the skin surface and, among them, the most commonly used are ethanol, cyclodextrins and triclosan. These substances act through a variety of mechanisms, such as depriving the environment of substances needed for bacterial growth or inhibiting bacterial enzymes needed for sweat degradation reactions.

Tannins are a large group of natural, complex, water-soluble phenolic substances which, in addition to the classical phenol reactions, precipitate alkaloids and other proteins, forming water insoluble complexes water with these compounds.

The tannins act on plant defense by inhibiting attack of vertebrate or invertebrate herbivores to them, since they reduce palatability, impair digestion and produce toxic compounds from their hydrolysis.

Tannins have 3 general characteristics: complexation with metal ions, antioxidant and scavenging of free radicals activities and complexation with macromolecules.

They are divided into two major groups, the condensed and hydrolysable groups, featuring various industrial applications, such as leather tanning, paint manufacturing, production of flocculants and coagulants for water treatment and development of astringent flavor of wines, fruit juices, teas and other beverages.

There are not many deodorant and antiperspirant formulations exhibiting absorption, reduction and/or elimination activity of axillary odors, which present a natural compound as the active principle for these actions.

With the growing demand for products with ecological appeal that stimulate reflection on sustainability, a need in cosmetic field has occurred for products with these characteristics.

So, there is an ongoing need for developing compositions with deodorant and antiperspirant action containing active principles of natural origin which are safe and effective for the proposed action, to meet the growing need for environmentally friendly products.

### DESCRIPTION OF THE INVENTION

The Applicant, looking for an alternative to common deodorants and antiperspirants and focusing on the growing need for environmentally appealing products, developed safe and effective deodorant and antiperspirant compositions containing a natural compound as the active principle.

Therefore, the invention relates to a DEODORANT and ANTIPERSPIRANT COMPOSITION, characterised by comprising natural tannins and one or more cosmetically acceptable adjuvant compounds.

The compositions of the present invention provide effective deodorant and antiperspirant action and comprise, as active principle, tannins obtained from natural origin, e.g. by a specific extraction process.

The tannins used as active principles in the composition of this invention may be obtained from any natural source containing such compounds, which are not merely limited to the cited examples. There are several plant species that have tannins in their compositions, such as quebracho, barbatimao, cohoba tree, mangrove, sweet chestnut, american chestnut, mastic tree, cashew, black jurema, mimosa, etc.

Certain plant tannins can be used from commercial powder presentations, such as PHENOTAN BS, sold by the company TANAC S.A., or SETA SUPERSOL LIGHT, sold by the company SETA S.A., both black wattle extracts with more than 70% of tannings.

Appropriate the tannin active principles of the composition of this invention are obtained by extraction processes, which comprise the extraction of macerated product, for example, in concentrate ethanol, from any suitable natural source containing tannins, thus obtaining a hydroalcoholic extract with significant concentration of tannins.

Within a particular embodiment of the invention, after extraction, the extract partition with ethyl acetate and butanol provides three distinct fractions with varying concentrations of tannins. Any of these fractions, besides of the hydroalcoholic extract, can be used as active principles comprised in the deodorant and antiperspirant compositions of this invention.

The compositions of the invention, without excluding other alternatives, comprise active principle concentrations from 0.1 to 99% of the formulation, preferably from 5 to 10%, regarding tanning agents.

The compositions of the present invention are useful in the absorption, reduction and/or elimination of axillary odors from perspiration and can be presented in the usual cosmetic forms, for example stick, roll-on, aerosol, spray, cream, ointment, gel, powder, liquid, lotion, etc. The formulations can be also presented as oil-in-water emulsions, water-in-oil emulsions, multiple emulsions or foaming products.

Adjuvant compounds to the products of the invention appropriate to the desired purpose are known, for example, thickeners, emollients, surfactants, emulsifiers, preservatives, propellant, vehicle, filler, perfume, solvent, antiseptic, germicide, bactericide, bacteriostatic, fungistatic, suspending agents, hardening agents, antioxidants, pigments, dyes, anti-syneresis agents, waxy agents, coupling agents, chelating agents, buffers, etc.

In a particular embodiment of the invention, the deodorants/antiperspirant compositions contain other active principles, for example, antiperspirant metallic salts or astringent metallic salts, such as organic and inorganic aluminum, zinc and zirconium salts or mixtures thereof. In more detail, the following additional optional salts for the product of the invention can be cited as adequate:

aluminum salts, such as chloride, acetate, bromide, nitrate, hydroxylactate, alumen, propylene glycol complexes of aluminum hydrochloride, aluminum hydrobromide, aluminum halides, aluminium halohydrates (e.g., aluminum hydrochloride), aluminum hydroxyhalides and mixtures thereof;

zirconium salts, such as chloride, acetate, bromide and nitrate, zirconium hydroxychloride, propylene glycol complexes of zirconium chloroxide, aluminium and zirconium pentahydxochloride, zirconium oxyhalides, zirconium hydroxyhalides and mixtures thereof;

other cosmetically acceptable salts such as zinc or magnesium.

In a particular embodiment of the invention, the compositions comprise as adjuvants:

from 0.05 to 0.1% of a chelating agent,

from 0.1 to 16% of a wetting compound

from 0.1 to 0.2% of preservative agents

from 0.1 to 17% of silicones

Among particular embodiments of the invention, the deodorant antiperspirant compositions of the present invention can be configured as absorbent products (for example, diapers, feminine napkins, nursing absorbent breast pads, wound bandages) and cosmetics (deodorant and antiperspirants per se, shampoos, moisturizing creams, sunscreens, soaps and toilet bars, depilation creams or waxes, etc.

Another aspect of the invention relates to processes for producing deodorant compositions, characterised by comprising the following steps:

(A) Mixing water, preservative, active principle and chelant agent, homogenizing them.

(B) Separately mixing the wetting agent with perfume, until homogenization.

(C) Adding the mixture obtained in step (B) to the mixture obtained in step (A) until homogenization.

(D) Mixing the silicones until homogenization.

(E) Slowly adding the mixture obtained in (C) over the mixture obtained in (D), at room temperature, with vigorous agitation, e.g. 1400-3000 rpm.

(F) Adjusting the pH to 6-6.5, when necessary.

The active principle used in this process can be of any natural origin, particularly tannins obtained from any natural source by a process comprising extraction and subsequent partition with ethyl acetate and butanol, and may be in the form of extract. or any fraction obtained form extract partition.

Additionally, another object of the present invention. comprises a method for the prevention and/or reduction of the perspiration odors that consists of applying or contacting the skin with an effective amount of deodorant and antiperspirant composition containing tannins as active principle, as it has been previously detailed.

### EXAMPLES

The following presented examples are merely illustrative of the realization of the present invention. These examples do not represent any limitation to the scope of this invention, which is determined by the claims that follows.

### Example 1

### Extraction of Tannins from Fresh Cashews

The chestnut from the fresh fruit was removed and discarded. The remainder of the fruit was sliced and macerated during 4 days in 96%: ethanol. Then, it was filtered through a Buchner funnel, obtaining a hydroalcoholic extract.

### Example 2

### Extract Partition

The hydroalcoholic extract obtained was partitioned with ethyl acetate and butanol to obtain specific fractions of tannins.

1 liter of the hydroalcoholic extract from the previous example was concentrated until removing all ethanol, and it was brought up to 550 mL with water, extracting with 500 mL of ethyl acetate, with further agitation and phase separation. Two distinct fractions were obtained after performing this procedure, one comprising ethyl acetate and the other an aqueous fraction.

The aqueous fraction was extracted with 500 mL of ethyl acetate, with subsequent stirring and phase separation, and the ethyl acetate fraction was combined, obtaining a fraction with 730 mg.

From the obtained aqueous fraction, a new extraction was carried out with 500 mL of butanol with agitation for separating the phases, again obtaining two fractions, a new aqueous fraction and a butanol fraction.

A new extraction was performed with the aqueous fraction, using 500 mL of butanol, agitating and separating the phases, thus obtaining an aqueous fraction with 79 g. The butanol portion was combined, thus obtaining a fraction with 6.7 g.

### Example 3

### Comparative Thin-Layer Chromatography (tlc)

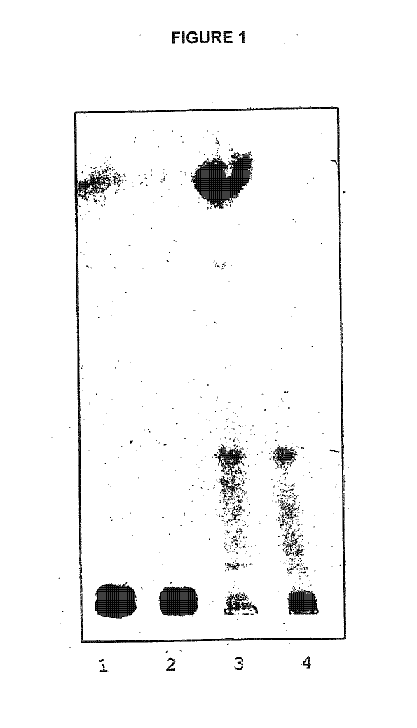

The fractions of examples 1 and 2 were subjected to thin-layer chromatography, obtaining the following results (see FIG. 1):

1—Hydroalcoholic extract

2—Aqueous Fraction

3—Ethyl Acetate Fraction

4—Butanol Fraction

Eluent: Ethyl acetate:formic acid:water (90:5:5)

Developer: 4-anisaldehyde

### Example 4

### Deodorant and Antiperspirant Natural Composition

The following composition was formulated from the extract obtained in example 1:

| | |
|---|---|
| Natural Deodorant and Antiperspirant | % |

Phase A

| | |
|---|---|
| DEMINERALIZED WATER qsp. | 100.00 |
| GLYDANT PLUS LIQUID | 0.20 |
| AQUEOUS FRACTION OF HYDROALCOHOLIC | 5.00 |
| EXTRACT CONTAINING TANNINS | |
| DISODIUM EDTA | 0.05 |

PHASE B

| | |
|---|---|
| PROPYLENE GLYCOL | 16.00 |
| PERFUME | 0.00 |

PHASE C

| | |
|---|---|
| CYCLOPENTASILOXANE (DC 245) | 7.00 |
| CYCLOPENTASILOXANE (E) PEG/PPG-18/18 | 10.00 |
| DIMETHICONE (DC 5225 c) | |

### Example 5

### Efficacy Assay

A cream comprising 5% of tannin extract, from example 4, was used in tests with 20 volunteers who rated the deodorant and antiperspirant action using the following parameters: good, very good or regular, as shown in the table below:

| | |
|---|---|
| Good | 17 volunteers |
| Very good | 2 volunteers |
| Regular | 1 volunteer |

1. A deodorant and antiperspirant composition, comprising natural tannins and at least one cosmetically acceptable adjuvant.

2. The composition according to claim 1, wherein the tannin is in the form of hydroalcoholic extract or one or more fractions obtained from the partition of said extract with ethyl acetate and butanol.

3. The composition according to claim 1, wherein the tannins are from a plant selected from the group consisting of: quebracho, barbatimao, cohoba tree, mangrove, sweet chestnut, american chestnut, mastic tree, cashew, black jurema and mimosa.

4. The composition according to claim 1, wherein the active principle is present at a concentration of 0.1 to 99% by mass of tannings.

5. The composition according to claim 1, wherein the active principle is present at a concentration of 5 to 10% of the formulation.

6. The composition according to claim 1, wherein, the adjuvant is selected from the group consisting of: a chelating compound, a humectant compound, preservatives, silicones, demineralized water, and combinations thereof.

7. The composition according to claim 6, wherein the chelating agent is present in mass concentrations between 0.01 to 0.05%, the wetting agent between 0.1 and 16%, the preservative between 0.1 and 0.2% and one or more silicones between 0.1 and 17%.

8. The composition according to claim 1 presented in a form selected from the group consisting of: stick, roll-on, aerosol, spray, cream, ointment, gel, powder and lotion.

9. The composition according to claim 1, presented in the form of an absorbent product, selected from the group consisting of: diapers, feminine napkins, nursing absorbent breast pads and wound plasters.

10. The composition according to claim 1, presented as a cosmetic selected from the group consisting of: shampoos, moisturizing creams, sunscreens, soaps, toilet bars, depilation creams and waxes.

11. A process for production of a deodorant and antiperspirant composition, comprising the following steps:
(A) mixing water, preservative, one or ore tannin, a chelanting agent, and homogenizing them;
(B) separately mixing a wetting agent with perfume, until Homogenization;
(C) adding the mixture obtained in step (B) to the mixture obtained in step (A) until homogenization;
(D) mixing one or more silicones until homogenization;
(E) slowly adding the mixture obtained in (C) to the mixture obtained in (D), at room temperature, with vigorous agitation;
(F) adjusting the pH to 6-6.5, when necessary.

12. The process, according to claim 11, wherein the tannin is in the form of hydroalcoholic extract or a fraction obtained by partition of the extract with ethyl acetate and butanol.

13. A method for the prevention and/or reduction of perspiration odors, comprising applying or contacting the skin surface with an effective amount of a composition defined in claim 1.
